(19) United States
(12) Patent Application Publication
(10) Pub. No.: US 2008/0119498 A1
(43) Pub. Date: May 22, 2008

(54) Therapeutic Agent for Pruritus Comprising P38 Map Kinase Inhibitor as the Active Ingredient

(76) Inventors: Masatomo Kato, Ikoma-shi (JP); Tomoko Oda, Ikoma-shi (JP); Daisuke Shii, Ikoma-shi (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/791,871

(22) PCT Filed: Dec. 6, 2005
(86) PCT No.: PCT/JP05/22321

§ 371 Date: May 30, 2007

(30) Foreign Application Priority Data
Dec. 6, 2004 (JP) 2004-352616

(51) Int. Cl. A61K 31/513; A61K 31/4439

(52) U.S. Cl. 514/269; 514/341

(57) ABSTRACT

An object of the present invention is to find a novel pharmacological effect (a medicinal use) of a p38 MAP kinase inhibitor. Because of having an excellent antipruritic effect, a p38 MAP kinase inhibitor is useful as a therapeutic agent for pruritus of any types such as ocular pruritus, skin pruritus and systemic pruritus.

### TECHNICAL FIELD

The present invention relates to a therapeutic agent for pruritus comprising a p38 MAP kinase inhibitor as an active ingredient.

### BACKGROUND ART

h respect to pruritus, a pruritus receptor present in the dermoepidermal junction of the skin or the mucosa is stimulated with a mediator (pruritus-inducing substance), and this stimulation is transmitted to the central nerve and felt as pruritus. As the mediator that induces pruritus, for example, histamines, platelet activating factors, kinins, bile salts, substance P, prostaglandins and the like are widely known. A mediator such as histamine released from mast cells and the like is presumably involved in pruritus caused by allergic factors, and an antihistaminic agent is known to exhibit a more potent effect than an antiallergic agent.

As pruritus, for example, ocular pruritus, skin pruritus, otic pruritus, nasal pruritus, systemic pruritus and the like which are caused in humans or animals are known. Ocular pruritus is a disease with itchy eyes, eyelids, eyelid edges and the like caused by foreign substances (such as pollen, dust, mites, molds, pet's hair, contact lens and cosmetics), dryness of eyes, injury thereof and the like. Further, scratching eyes may lead to conjunctival hyperemia or conjunctival injury in some cases. In this way, pruritus not only lowers the daily quality of life of patients, but also becomes a cause of worsening the symptoms by scratching.

On the other hand, p38 mitogen-activated protein kinase (p38 MAP kinase) is one of the enzymes mediating the mechanism of apoptosis and is known as a protein binding to a cytokine-suppressive anti-inflammatory drug. JP-A-2002-97189 describes that a compound such as 4-(4-fluorophenyl)-2-(4-hydroxyphenyl)-5-(4-pyridyl)-1H-imidazole or 4-(4-fluorophenyl)-2-(4-methylsulfinylphenyl)-5-(4-pyridyl)-1H-imidazole is effective in the treatment of inflammatory cytokine-mediated diseases such as chronic rheumatoid arthritis, osteoarthritis and gouty arthritis. Further, JP-T-2000-503304 describes that a compound such as trans-4-[4-(4-fluorophenyl)-5-(2-methoxy-4-pyrimidinyl)-1H-imidazole-1-yl]cyclohexanol is effective in the treatment of inflammatory cytokine-mediated diseases such as psoriatic arthritis, chronic rheumatoid arthritis, sunburn and conjunctivitis. JP-T-2001-522357 describes that a substituted imidazole compound having a p38 MAP kinase inhibitory effect is useful for inflammatory cytokine-mediated diseases such as rheumatoid arthritis, inflammatory bowel diseases, septic shock and osteoporosis.

However, an antipruritic effect of a compound having a p38 MAP kinase inhibitory effect has not been studied at all in these patent documents.

### DISCLOSURE OF THE INVENTION

### Problems to be Solved

As described above, a p38 MAP kinase inhibitor has various pharmacological effects as a medicinal agent. However, it is an interesting subject to find an effectiveness against pruritus as another new pharmacological effect.

### Means of Solving Problems

The present inventors have made intensive studies in order to find a new medicinal use of the p38 MAP kinase inhibitor, and as a result, they found that in an ocular pruritus inhibition test using histamine-induced models and platelet activating factor-induced models, the p38 MAP kinase inhibitor exhibits an excellent antipruritic effect by directly acting on the peripheral nerve terminals and controlling transmission of pruritus signal in neurons, and thus the present invention has been accomplished.

That is, the present invention is directed to:

(1) a therapeutic agent for pruritus comprising a p38 MAP kinase inhibitor as an active ingredient;

(2) a therapeutic agent for pruritus comprising at least one compound selected from the group consisting of 4-(4-fluorophenyl)-2-(4-hydroxyphenyl)-5-(4-pyridyl)-1H-imidazole, trans-4-[4-(4-fluorophenyl)-5-(2-methoxy-4-pyrimidinyl)-1H-imidazole-1-yl]cyclohexanol, 4-(4-fluorophenyl)-2-(4-methylsulfinylphenyl)-5-(4-pyridyl)-1H-imidazole and salts thereof as an active ingredient;

(3) the therapeutic agent for pruritus according to the above (1) or (2), wherein the pruritus is ocular pruritus;

(4) the therapeutic agent for pruritus according to any one of the above (1) to (3), wherein the dosage form thereof is a liquid;

(5) the therapeutic agent for pruritus according to the above (4), wherein the liquid is an eye drop;

(6) the therapeutic agent for pruritus according to the above (5), wherein the concentration of the active ingredient in the eye drop is in the range of from 0.01 to 3% (w/v);

(7) the therapeutic agent for pruritus according to any one of the above (1) to (3), wherein the dosage form is an external preparation;

(8) the therapeutic agent for pruritus according to the above (7), wherein the external preparation is an ointment; and

(9) the therapeutic agent for pruritus according to the above (8), wherein the ointment is an eye ointment;

In the present invention, the pharmaceutically acceptable salts are not particularly limited and examples thereof include salts with an inorganic acid such as hydrochloric acid, nitric acid or sulfuric acid, salts with an organic acid such as acetic acid, fumaric acid, maleic acid, succinic acid or tartaric acid, and salts with an alkali metal or an alkaline earth metal such as sodium, potassium, lithium or calcium, and the like. More preferred salts are sodium salts, lithium salts and potassium salts. Further, quaternary ammonium salts, hydrates and solvates are also included in the pharmaceutically acceptable salts of the present invention. Further, geometric isomers, optical isomers, tautomers, polymorphisms and the like are also included in the scope of the present invention.

In the present invention, the p38 MAP kinase inhibitor is not particularly limited as long as it is a compound having a p38 MAP kinase inhibitory activity. Examples thereof include compounds described in patent publications such as JP-A-2002-97189, JP-T-2000-503304, JP-T-2001-522357, JP-T-2003-535023, JP-T-2001-506266, JP-T-9-508123, International Publication No. WO 01/56553, International Publication No. WO 93/14081, International Publication No. WO 01/35959, International Publication No. WO 03/68229, International Publication No. WO 03/85859, JP-T-2002-534468, JP-T-2001-526222, JP-T-2001-526223, U.S. Pat. No. 6,344,476, International Publication No. WO 03/99811, International Publication No. WO 03/99796, JP-T-2004-506042, International Publication No. WO 04/60286, JP-T-2002-363179, JP-T-2004-107358, U.S. Pat. No. 5,670,527, U.S. Pat. No. 6,096,753, International Publication No. WO 01/42189 and International Publication No. WO 00/31063. Preferred examples thereof include 4-(4-fluorophenyl)-2-(4-hydroxyphenyl)-5-(4-pyridyl)-1H-imidazole (SB-202190), trans-4-[4-(4-fluorophenyl)-5-(2-methoxy-4-pyrimidinyl)-1H-imidazole-1-yl]cyclohexanol (SB-239063), 4-(4-fluorophenyl)-2-(4-methylsulfinylphenyl)-5-(4-pyridyl)-1H-imidazole (SB-203580), 4-(4-fluorophenyl)-5-(2-methoxypyrimidine-4-yl)-1-(piperidine-4-yl)imidazole (SB-242235), 4-(4-fluorophenyl)-2-(4-hydroxy-1-butynyl)-1-(3-phenylpropyl)-5-(4-pyridyl)imidazole (RWJ-67657), 4-(4-fluorophenyl)-1-(piperidine-4-yl)-5-(4-pyridyl)imidazole (HEP-689), (S)-2-(2-amino-3-phenylpropylamino)-1-methyl-5-(2-naphthyl)-4-(4-pyridyl)pyrimidine-6-one (AMG-548), 2-chloro-4-(4-fluoro-2-methylanilino)-2′-methylbenzophenone (EO-1606), 3-(4-chlorophenyl)-5-(1-hydroxyacetyl-piperidine-4-yl)-4-(pyrimidine-4-yl)pyrazole (SD-06), 5-(2,6-dichlorophenyl)-2-(2,4-difluorophenylthio) pyrimido[3,4-b]pyridazine-6-one (VX-745), 4-acetylamino-N-tert-butylbenzamide (CPI-1189), N-[3-tert-butyl-1-(4-methylphenyl)pyrazole-5-yl]-N′-[4-(2-morpholinoethoxy)-1-naphthyl]urea (Dramapimod), 2-benzamide-4-[2-ethyl-4-(3-methylphenyl)thiazole-5-yl]pyridine (TAK-715), SCIO-469, VX-702, GSK-681323, PS-540446, SC-80036, AVE-9940, RO-320-1195, SB-281832, SCIO-323, KC-706 and Semapimod.

With respect to pruritus, a pruritus receptor present in the peripheral nerve terminals is stimulated with a mediator (pruritus-inducing substance) such as histamine to cause excitation of neurons, whereby the stimulation is transmitted to the central nerve and felt as pruritus. On the other hand, the function of a MAP kinase pathway in neurons has been drawing attention, and MAP kinase is known to be activated also by electrical excitation. Accordingly, the present inventors hypothesized that a p38 MAP kinase inhibitor acts on the peripheral nerve involved in pruritus such as sensory nerve and inhibits the transmission of pruritus, and made examination using typical compounds known as a p38 MAP kinase inhibitor.

As is clear from the results of an ocular pruritus inhibition test using histamine-induced models and platelet activating factor-induced models described later, the therapeutic agent for pruritus according to the present invention directly acts on the peripheral nerve terminals and controls transmission of pruritus signal in neurons, therefore, it exhibits an excellent antipruritic effect on pruritus caused by any factors. Thus, the therapeutic agent can be expected to exhibit a therapeutic and/or preventive effect on pruritus such as ocular pruritus, skin pruritus, otic pruritus, nasal pruritus and systemic pruritus which are caused in humans or animals. Particularly preferably, the therapeutic agent is used as a therapeutic agent for ocular pruritus.

The ocular pruritus in the present invention is a disease with itchy eyes, eyelids, eyelid edges and the like caused by pollen, dust, mites, molds, pet's hair, contact lens, cosmetics, injury of eyes and the like, and includes diseases caused by various factors.

The therapeutic agent for pruritus according to the present invention can be formulated into a single preparation or a mixed preparation by adding a pharmaceutically acceptable additive, as needed, with the use of a widely used technique.

Further, the therapeutic agent for pruritus according to the present invention can be administered either parenterally or orally. Examples of an oral preparation include liquid preparations for oral administration (such as elixirs, syrups, pharmaceutically acceptable aqueous preparations, suspensions and emulsions), solid preparations for oral administration (such as tablets (including sublingual tablets and orally disintegrating tablets), pills, capsules (including hard capsules, soft capsules, gelatin capsules and microcapsules), powders, granules and lozenges) and the like. Examples of a parenteral preparation include liquid preparations (for example injections (subcutaneous injections, intravenous injections, intramuscular injections, intraperitoneal injections and intravenous infusions and the like), eye drops (such as aqueous eye drops (aqueous ophthalmic solutions, aqueous suspended ophthalmic solutions, viscous ophthalmic solutions, solubilized ophthalmic solutions and the like), and non-aqueous eye drops (non-aqueous ophthalmic solutions, non-aqueous suspended ophthalmic solutions and the like)) and the like), external preparations (such as ointments (eye ointments and the like), gels, creams, poultices, adhesive preparations and liniments), nebulas, inhalants, sprays, nasal drops, suppositories (such as rectal suppositories and vaginal suppositories) and the like. These preparations may be a controlled release preparation such as an immediate release preparation or a sustained release preparation. These preparations can be prepared by a known method, for example, a method described in the Japanese Pharmacopoeia or the like.

The liquid preparation for oral administration as an oral preparation is prepared by, for example, dissolving, suspending or emulsifying the active ingredient in a generally used diluent (such as purified water, ethanol or a mixed solution thereof). The liquid preparation for oral administration may further contain a moistening agent, a suspending agent, an emulsifier, a sweetener, a flavor, an aromatic, a preservative, a buffer or the like.

The solid preparation for oral administration as an oral preparation is prepared by, for example, mixing the active ingredient with an excipient (such as lactose, mannitol, glucose, microcrystalline cellulose or starch), a binder (such as hydroxypropyl cellulose, polyvinyl pyrrolidone or magnesium aluminometasilicate), a disintegrant (such as fibrous calcium glycolate), a lubricant (such as magnesium stearate), a stabilizer, a solubilizing agent (such as glutamic acid or asparatic acid) or the like in accordance with a standard method. Also, if necessary, the solid preparation for oral administration may be coated with a coating agent (such as sucrose, gelatin, hydroxypropyl cellulose or hydroxypropylmethyl cellulose phthalate). The coating layer may consist of two or more layers.

The external preparation as a parenteral preparation is prepared by a known method or a commonly used recipe. For example, an ointment is prepared by levigating or fusing the active ingredient with a base. The ointment base is selected from ointment bases which are known or commonly used. For example, those selected from higher fatty acids or higher fatty acid esters (such as adipic acid, myristic acid, palmitic acid, stearic acid, oleic acid, adipic acid esters, myristic acid esters, palmitic acid esters, stearic acid esters and oleic acid esters), waxes (such as beeswax, spermaceti and ceresin), surfactants (such as polyoxyethylene alkyl ether phosphoric acid esters), higher alcohols (such as cetanol, stearyl alcohol and cetostearyl alcohol), silicone oils (such as dimethyl polysiloxane), hydrocarbons (such as hydrophilic soft paraffin, white soft paraffin, purified lanolin and liquid paraffin), glycols (such as ethylene glycol, diethylene glycol, propylene glycol, polyethylene glycol and macrogol), vegetable oils (such as castor oil, olive oil, sesame oil and turpentine oil), animal oils (such as mink oil, egg yolk oil, squalane and squalene), water, absorption accelerators and rash-preventing agents are used alone or as a mixture of two or more. The ointment may further contain a humectant, a preservative, a stabilizer, an antioxidant, a flavoring agent or the like.

The gel is prepared by, for example, fusing the active ingredient with a base. The gel base is selected from gel bases which are known or commonly used. For example, those selected from lower alcohols (such as ethanol and isopropyl alcohol), gelling agents (such as carboxymethyl cellulose, hydroxyethyl cellulose, hydroxypropyl cellulose and ethyl cellulose), neutralizing agents (such as triethanolamine and diisopropanolamine), surfactants (such as polyethylene glycol monostearate), gums, water, absorption accelerators and rash-preventing agents are used alone or as a mixture of two or more. The gel may further contain a preservative, an antioxidant, a flavoring agent or the like.

The cream is prepared by, for example, fusing or emulsifying the active ingredient with or in a base. The cream base is selected from cream bases which are known or commonly used. For example, those selected from higher fatty acid esters, lower alcohols, hydrocarbons, polyhydric alcohols (such as propylene glycol and 1,3-butylene glycol), higher alcohols (such as 2-hexyldecanol and cetanol), emulsifiers (such as polyoxyethylene alkyl ethers and fatty acid esters), water, absorption accelerators and rash-preventing agents are used alone or as a mixture of two or more. The cream may further contain a preservative, an antioxidant, a flavoring agent or the like.

The poultice is prepared by, for example, fusing the active ingredient with a base to form a kneaded product, and spreading and coating the kneaded product on a support. The poultice base is selected from poultice bases which are known or commonly used. For example, those selected from viscous agents (such as polyacrylic acid, polyvinyl pyrrolidone, gum arabic, starch, gelatin and methyl cellulose), moistening agents (such as urea, glycerin and propylene glycol), fillers (such as kaolin, zinc oxide, talc, calcium and magnesium), water, solubilizing agents, tackifiers and rash-preventing agents are used alone or as a mixture of two or more. The poultice may further contain a preservative, an antioxidant, a flavoring agent or the like.

The adhesive preparation is prepared by, for example, fusing the active ingredient with a base and spreading and coating the resulting product on a support. The adhesive preparation base is selected from adhesive preparation bases which are known or commonly used. For example, those selected from polymer bases, oils and fats, higher fatty acids, tackifiers and rash-preventing agents are used alone or as a mixture of two or more. The adhesive preparation may further contain a preservative, an antioxidant, a flavoring agent or the like.

The liniment is prepared by, for example, dissolving, suspending or emulsifying the active ingredient in one or more media selected from water, alcohols (such as ethanol and polyethylene glycol), higher fatty acids, glycerins, soaps, emulsifiers, suspending agents and the like. The liniment may further contain a preservative, an antioxidant, a flavoring agent or the like.

The nebula and spray are prepared by a known or commonly used recipe. These preparations may contain, for example, a stabilizer such as sodium bisulfite and a buffer that imparts isotonicity, for example, a tonicity agent such as sodium chloride, sodium citrate or citric acid in addition to the generally used diluent.

The inhalant includes aerosols, inhalation powders and inhalation liquids. The inhalation liquid may be in the form in which it is used by dissolving or suspending in water or another appropriate medium immediately before use. The inhalation liquid is prepared by using an antiseptic agent (such as benzalkonium chloride or paraben), a coloring agent, a buffer (such as sodium phosphate or sodium acetate), a tonicity agent (such as sodium chloride or concentrated glycerin), a viscous agent (such as carboxy vinyl polymer), an absorption accelerator or the like as needed. The inhalation powder is prepared by using a lubricant (such as stearic acid or a salt thereof), a binder (such as starch or dextrin), an excipient (such as lactose or cellulose), a coloring agent, an antiseptic agent (such as benzalkonium chloride or paraben), an absorption accelerator or the like as needed. When the inhalation liquid is administered, a sprayer (such as an atomizer or a nebulizer) is commonly used, and when the inhalation powder is administered, an inhalator for powder preparations is commonly used.

The injection as a parenteral preparation includes solutions, suspensions, emulsions and injections in the solid form to be used by dissolving or suspending in a solvent immediately before use. The injection is used by, for example, dissolving, suspending or emulsifying the active ingredient in a solvent. As the solvent, for example, distilled water for injection, a physiological saline solution, a vegetable oil, propylene glycol, polyethylene glycol, an alcohol such as ethanol, or a mixture thereof is used. The injection may further contain a stabilizer, a solubilizing agent (such as glutamic acid, aspartic acid or polysorbate 80 (registered trademark)), a suspending agent, an emulsifier, a soothing agent, a buffer, a preservative or the like. It is preferred that the injection is prepared through sterilization in a final step, or prepared according to a sterile process. In the case of a sterile solid preparation, it is also possible to prepare, for example, a lyophilized preparation, which is used by dissolving it before use in sterilized or sterile distilled water or another solvent for injection.

In order to use the therapeutic agent for pruritus according to the present invention as a therapeutic agent for ocular pruritus, the preferred dosage form thereof is an eye drop, an eye ointment, a tablet or the like, and more preferred dosage form thereof is an eye drop or an eye ointment. These preparations can be prepared by a widely used technique. For example, the eye drop can be prepared by appropriately adding a tonicity agent, a buffer, a pH adjusting agent, a solubilizer, a viscous agent, a stabilizer, a preservative or the like as an additive. Further, by adding a pH adjusting agent, a viscous agent, a dispersant or the like and suspending a medicament, a stable eye drop can also be obtained.

Examples of the tonicity agent include glycerin, propyleneglycol, sodium chloride, potassium chloride, sorbitol, mannitol and the like.

Examples of the buffer include phosphoric acid, phosphates, citric acid, acetic acid, ε-aminocaproic acid and the like.

Examples of the pH adjusting agent include hydrochloric acid, citric acid, phosphoric acid, acetic acid, sodium hydroxide, potassium hydroxide, boric acid, borax, sodium carbonate, sodium hydrogencarbonate and the like.

Examples of the solubilizer include polysorbate 80, polyoxyethylene hydrogenated castor oil 60, macrogol 4000 and the like.

Examples of the viscous agent and the dispersant include cellulose polymers such as hydroxypropylmethyl cellulose and hydroxypropyl cellulose, polyvinyl alcohols, polyvinyl pyrrolidone and the like. Further, examples of the stabilizer include edetic acid, sodium edetate and the like.

Examples of the preservative (antiseptic agent) include widely used sorbic acid, potassium sorbate, benzalkonium chloride, benzethonium chloride, methyl p-oxybenzoate, propyl p-oxybenzoate, chlorobutanol and the like. These preservatives can also be used in combination.

In the case where the therapeutic agent for pruritus according to the present invention is an eye drop, the pH thereof is preferably adjusted to 4.0 to 8.5 and the osmotic pressure rate thereof is preferably adjusted to around 1.0.

The present invention also relates to a method of treating pruritus comprising administering an effective amount of the p38 MAP kinase inhibitor to a patient.

The dose of the p38 MAP kinase inhibitor can be properly selected depending on the symptoms, age, dosage form and the like. In the case of an oral preparation, it may be administered once to several times (for example, 1 to 3 times) a day in an amount of preferably from 1 mg to 100 mg, more preferably from 5 mg to 30 mg. In the case of an eye drop, an eye drop at a concentration of preferably from 0.001 to 10% (w/v), more preferably from 0.01 to 3% (w/v) may be instilled in an amount of one to several drops at a time once to several times (for example, 1 to 8 times) a day. In the case of an eye ointment, an eye ointment at a concentration of preferably from 0.001 to 10% (w/w), more preferably from 0.01 to 3% (w/w) may be applied once to several times (for example, 1 to 4 times) a day.

Of course, as described above, because the dose varies depending on the various conditions, a dose less than the above-mentioned dose is sufficient in some cases, and a dose exceeding the above-mentioned range is needed in some cases.

### ADVANTAGE OF THE INVENTION

When an ocular pruritus inhibition test was carried out, p38 MAP kinase inhibitors such as 4-(4-fluorophenyl)-2-(4-hydroxyphenyl)-5-(4-pyridyl)-1H-imidazole hydrochloride, trans-4-[4-(4-fluorophenyl)-5-(2-methoxy-4-pyrimidinyl)-1H-imidazole-1-yl]cyclohexanol and 4-(4-fluorophenyl)-2-(4-methylsulfinylphenyl)-5-(4-pyridyl)-1H-imidazole hydrochloride exhibited an excellent antipruritic effect in histamine-induced models or platelet activating factor-induced models, therefore, they are useful as a therapeutic agent for pruritus of any types such as ocular pruritus, skin pruritus and systemic pruritus.

### BEST MODE FOR CARRYING OUT THE INVENTION

Hereinafter, a pharmacological test and preparation examples will be described, however, these examples are described for the purpose of understanding the present invention better and are not meant to limit the scope of the present invention.

### [Pharmacological Test]

By using histamine-induced models and platelet activating factor-induced models, the ocular pruritus inhibition activity of p38 MAP kinase inhibitors were studied. With regard to the p38 MAP kinase inhibitors, 4-(4-fluorophenyl)-2-(4-hydroxyphenyl)-5-(4-pyridyl)-1H-imidazole hydrochloride (hereinafter referred to as “Compound A”), trans-4-[4-(4-fluorophenyl)-5-(2-methoxy-4-pyrimidinyl)-1H-imidazole-1-yl]cyclohexanol (hereinafter referred to as “Compound B”) and 4-(4-fluorophenyl)-2-(4-methylsulfinylphenyl)-5-(4-pyridyl)-1H-imidazole hydrochloride (hereinafter referred to as “Compound C”) were used as the typical examples.

### (1) Ocular Pruritus Inhibition Activity to Histamine-induced Models

### (Experimental Method)

Compound A was dissolved in a 5.0% Tween 80/physiological saline solution at a concentration of 0.1% (w/v), and Compound B was suspended in a 5.0% Tween 80/physiological saline solution at a concentration of 0.1% (w/v), whereby the respective test compound solutions were prepared.

Each test compound solution was instilled into both eyes of 5-week-old male Hartley guinea pigs in an amount of 10 μl per eye. At 15 minutes thereafter, each test compound solution of the same concentration was instilled again (twice in total). As a control, a 5.0% Tween 80/physiological saline solution was used.

After 15 minutes from the second instillation of each test compound solution, a physiological saline solution in which histamine was dissolved at 1.0% (w/v) was instilled into both eyes of the above guinea pigs in an amount of 10 μl per eye to induce eye scratching behavior.

The behavior of the guinea pigs after the instillation of histamine was videotaped, and the ocular pruritus was evaluated for each eye by counting the number of times of a series of behaviors of scratching the eye with the hind paws. In Table 1, the average of the number of eye scratching times for 30 minutes after the instillation of histamine and the average of the eye scratching behavior inhibition ratio are shown. Each group consisted of 8 eyes.

Eye scratching behavior inhibition ratio (%)=100−(number of eye scratching times for test compound)/(number of eye scratching times for control)×100

TABLE 1

| | Number of eye | |
|---|---|---|
| | scratching | Eye scratching behavior |
| | times (times) | inhibition ratio (%) |
| Control | 6.1 | — |
| Compound A (0.1%) | 1.4 | 77.0 |
| Compound B (0.1%) | 3.9 | 36.1 |

### (Experimental Results)

Table 1 shows that the number of eye scratching times of guinea pigs to which Compound A or Compound B was instilled was significantly decreased in comparison with the control. Therefore, it was confirmed that the p38 MAP kinase inhibitors have an excellent ocular pruritus inhibition activity.

### (2) Ocular Pruritus Inhibition Activity to Platelet Activating Factor-induced Models

### (Experimental Method)

Platelet activating factor-induced ocular pruritus evaluation models were prepared in accordance with the published method of Kato et al. (J. Ocul. Pharmacol. Ther. 2003; 19: 315-324). Compound C was dissolved in ultrapure water at a concentration of 0.01% or 0.1% (w/v), and each of the resulting solutions was instilled into both eyes of 5-week-old male Hartley guinea pigs in an amount of 10 μl per eye. At 15 minutes thereafter, each solution of Compound C of the same concentration was instilled again (twice in total). As a control, ultrapure water was used.

After 15 minutes from the second instillation of the solution of Compound C, a physiological saline solution in which a platelet activating factor was dissolved at 0.1% (w/v) was instilled into both eyes of the above guinea pigs in an amount of 10 μl per eye to induce eye scratching behavior.

The behavior of the guinea pigs after the instillation of the platelet activating factor was videotaped, and the ocular pruritus was evaluated for each eye by counting the number of times of a series of behaviors of scratching the eye with the hind paws. In Table 2, the average of the number of eye scratching times for 30 minutes after the instillation of the platelet activating factor and the average of the eye scratching behavior inhibition ratio are shown. Each group consisted of 8 eyes.

TABLE 2

| | Number of eye | |
|---|---|---|
| | scratching | Eye scratching behavior |
| | times (times) | inhibition ratio (%) |
| Control | 7.1 | — |
| Compound C (0.01%) | 0.9 | 87.3 |
| Compound C (0.1%) | 1.0 | 85.9 |

### (Experimental Results)

Table 2 shows that the number of eye scratching times of guinea pigs to which Compound C was instilled was significantly decreased in comparison with the control. Therefore, it was confirmed that the p38 MAP kinase inhibitor exhibits an ocular pruritus inhibition activity also in the platelet activating factor-induced models.

### PREPARATION EXAMPLES

Hereinafter, examples of typical preparations to be used in the present invention will be shown.

### 1. Eye Drop

An eye drop having the following formulation is prepared with a widely used method.

Formulation example 1
In 100 ml,

| | |
|---|---|
| Compound A | 100 mg |
| Concentrated glycerin | 500 mg |
| Polysorbate 80 | 1000 mg |
| Sodium dihydrogenphosphate dihydrate | q.s. |
| 1N Sodium hydroxide | q.s. |
| Hydrochloric acid | q.s. |
| Sterile purified water | q.s. |

In the same manner as in the Formulation example 1, an eye drop containing Compound A in an amount of 10 mg, 50 mg or 1000 mg in 100 ml can be prepared.

### 2. Eye Ointment

An eye ointment having the following formulation is prepared with a widely used method.

Formulation example 2
In 100 g,

| | |
|---|---|
| Compound C | 200 mg |
| Liquid paraffin | 10 g |
| White soft paraffin | q.s. |

By properly altering the amount of Compound C to be added, eye ointments of various concentrations can be prepared in the same manner as in the Formulation example 2.

1. A pharmaceutical composition for pruritus comprising a p38 MAP kinase inhibitor as an active ingredient and a pharmaceutical carrier.

2. A pharmaceutical composition for pruritus comprising at least one compound selected from the group consisting of 4-(4-fluorophenyl)-2-(4-hydroxyphenyl)-5-(4-pyridyl)-1H-imidazole, trans-4-[4-(4-fluorophenyl)-5-(2-methoxy-4-pyrimidinyl)-1H-imidazole-1-yl]cyclohexanol, 4-(4-fluorophenyl)-2-(4-methylsulfinylphenyl)-5-(4-pyridyl)-1H-imidazole and salts thereof as an active ingredient and a pharmaceutical carrier.

3. The pharmaceutical composition for pruritus according to claim 1 or 2, wherein the pruritus is ocular pruritus and an ophthalmic carrier.

4. The pharmaceutical composition for pruritus according to claim 1 or claim 2, wherein the dosage form thereof is a liquid and an ophthalmic carrier.

5. The pharmaceutical composition for pruritus according to claim 4, wherein the liquid is an eye drop.

6. The pharmaceutical composition for pruritus according to claim 5, wherein the concentration of the active ingredient in the eye drop is in the range of from 0.01 to 3% (w/v).

7. The pharmaceutical composition for pruritus according to claim 1 or claim 2, wherein the dosage form is an external preparation.

8. The pharmaceutical composition for pruritus according to claim 7, wherein the external preparation is an ointment.

9. The pharmaceutical composition for pruritus according to claim 8, wherein the ointment is an eye ointment.

10. A method of treating pruritus comprising administering an effective amount of a p38 MAP kinase inhibitor to a patient.

11. A method of treating pruritus comprising administering an effective amount of at least one compound selected from the group consisting of 4-(4-fluorophenyl)-2-(4-hydroxyphenyl)-5-(4-pyridyl)-1H-imidazole, trans-4-[4-(4-fluorophenyl)-5-(2-methoxy-4-pyrimidinyl)-1H-imidazole-1-yl]cyclohexanol, 4-(4-fluorophenyl)-2-(4-methylsulfinylphenyl)-5-(4-pyridyl)-1H-imidazole and salts thereof to a patient.

12. The method of treating pruritus according to claim 10 or 11, wherein the pruritus is ocular pruritus.

13. A method of treating pruritus comprising administering an effective amount of a p38 MAP kinase inhibitor in the form of a liquid to a patient.

14. A method of treating pruritus comprising administering an effective amount of a p38 MAP kinase inhibitor in the form of an eye drop to a patient.

15. The method of treating pruritus according to claim 14, wherein the concentration of the p38 MAP kinase inhibitor in the eye drop is in the range of from 0.01 to 3% (w/v).

16. A method of treating pruritus comprising administering an effective amount of a p38 MAP kinase inhibitor in the form of an external preparation to a patient.

17. The method of treating pruritus according to claim 16, wherein the external preparation is an ointment.

18. The method of treating pruritus according to claim 17, wherein the ointment is an eye ointment.

19-27. (canceled)
